(19) United States
(12) Patent Application Publication
(10) Pub. No.: US 2006/0229585 A1
(43) Pub. Date: Oct. 12, 2006

(54) DRUG DELIVERY TO THE CRYSTALLINE LENS AND OTHER OCULAR STRUCTURES

(75) Inventor: Gholam A. Peyman, Sun City, AZ (US)

Correspondence Address: WOOD, HERRON & EVANS, LLP, 2700 CAREW TOWER, 441 VINE STREET, CINCINNATI, OH, 45202, US

(73) Assignee: MINU, L.L.C., Pittsboro, NC (US)

(21) Appl. No.: 11/423,458

(22) Filed: Jun. 12, 2006

Related U.S. Application Data
(63) Continuation-in-part of application No. 11/103,283, filed on Apr. 11, 2005.

(51) Int. Cl. A61M 31/00

(52) U.S. Cl. 604/501; 604/20

(57) ABSTRACT

A method of delivering a drug or other compound to structures of the eye. A conduit through which a drug is introduced penetrates the eye so as to be located proximate an ocular structure, such as the crystalline lens or the retina, but does not penetrate the ocular structure. A drug is introduced adjacent the ocular structure using the conduit and is directed into the ocular structure using iontophoresis. In one embodiment, the conduit operates as one of the electrodes for the iontophoresis.

### RELATED APPLICATION

This application is a Continuation-in-Part of U.S application Ser. No. 11/103,283, filed Apr. 11, 2005 which is expressly incorporated by reference herein in its entirety.

### BRIEF DESCRIPTION OF DRAWINGS

FIG. 1 illustrates a side elevational view in cross-section of a human eye.

FIG. 1A is an enlarged view of the encircled area 1A of FIG. 1 showing a conduit to introduce agents into the lens in accordance with an embodiment of the invention.

FIG. 2 is an enlarged view of the eye similar to FIG. 1A showing introduction of agents into the lens in accordance with another embodiment of the invention.

FIG. 3 is an enlarged view of the encircled area 3 in FIG. 1 showing a conduit to introduce agents into the retina in accordance with an embodiment of the invention.

### DETAILED DESCRIPTION

A cataract refers to any opacity of the ocular crystalline lens. The normal crystalline lens is transparent, refractive, and provides adequate accommodation (shape change) to transmit and focus light on the retina at various distances. Accommodation in the human eye occurs through controlled changes in crystalline lens shape, thickness, and refractive surface placement relative to the cornea. With cataracts, the normally clear lens becomes cloudy, affecting the transmission of light through the lens and resulting in loss of visual acuity. Cataracts are most frequently associated with the normal aging process or pathology, but injury or mechanical violation of the ocular capsule surrounding the lens also causes cataract formation.

Because of the risk of inducing cataract formation, penetrating the lens capsule to introduce drugs or other agents into the lens has not been favorably considered.

One embodiment is an ocular drug delivery method by penetrating at least the outer capsule of the lens with a fine conduit that creates a self-sealing aperture when it is removed, then introducing a drug into the lens through this aperture. For example, a 30 gauge or higher needle, connected to a syringe, can be used. In various embodiments, the agent can be introduced into any part of the lens (e.g., anterior and/or posterior portion), into more than one location in the lens, and ultrasound can be performed to visualize placement of the conduit within the lens. The drug may be in a nanotechnology formulation.

In another embodiment a drug is delivered to the lens by electromotive drug administration, which does not require penetration of the lens capsule. A conduit is brought into proximity to the lens and a drug is introduced adjacent the lens. The conduit is configured to operate as an electrode for applying an electromotive force to the drugs released from the conduit adjacent the lens. A second electrode is strategically positioned either inside the body or outside the body so as to direct the drugs in a preferred direction, such as into the lens or within certain regions of the lens.

These methods provide a drug or other agent(s) directly to the lens while minimizing the risk of cataract formation. The methods also contain the agent within the lens, so that a higher than normal concentration of drug may be effectively delivered, or gene therapy may be provided directly to the lens with less concern for systemic toxicity, untoward treatment outcomes, etc. For example, a gene or gene fragment (promoter, etc) may be delivered in a vector using the inventive methods, allowing positive or negative regulation of lens epithelial cell proliferation, mortality, and/or integrity.

In the embodiment in which the lens is penetrated by the conduit, by regulating the depth of conduit penetration, the length of the conduit and/or by visualizing conduit placement, the agent can be delivered to the lens capsule, subcapsular epithelium, cortex, and/or nucleus. In a similar manner, in the embodiment in which the lens is not penetrated, the magnitude and the duration time of the electric current provides some control over placement of the agent within the lens.

The non-penetrating methodology may also be used to deliver a drug to other ocular structures. In another embodiment a drug is delivered to the retina by electromotive drug administration. A conduit is brought into proximity to the retina and a drug is introduced adjacent the retina. The conduit is configured to operate as an electrode for applying an electromotive force to the drugs released from the conduit adjacent the retina. A second electrode is strategically positioned either inside the body or outside the body so as to direct the drugs into the retina.

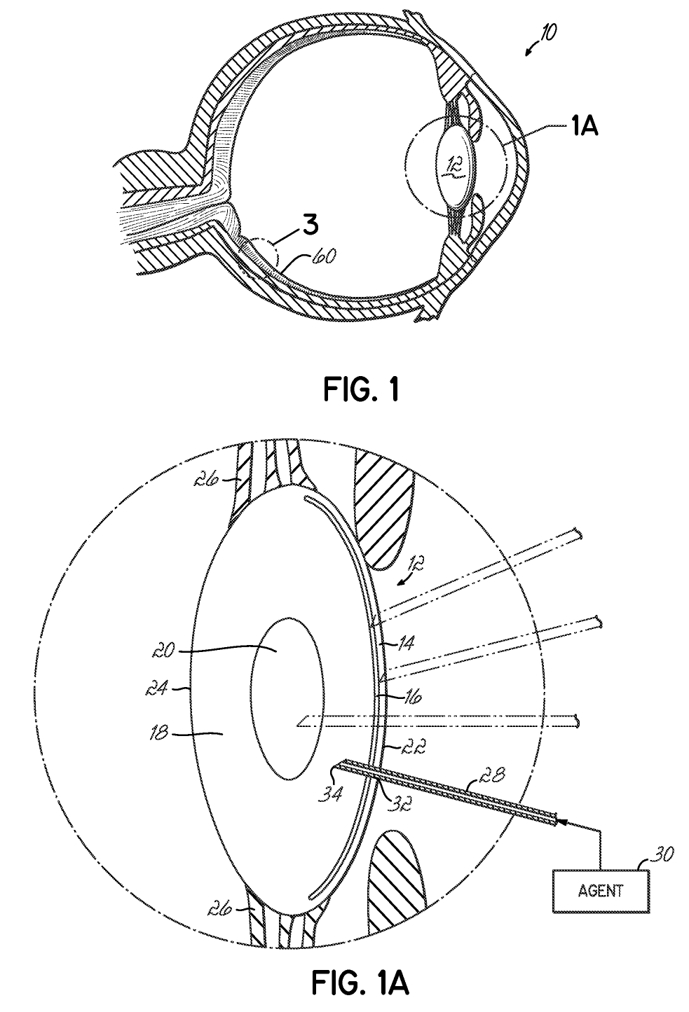

The inventive methods provide for the introduction of agents into the crystalline lens with a minimized risk of inducing cataract formation. As shown in FIGS. 1 and 1 A, the normal human eye 10 includes a transparent bioconvex crystalline lens 12, which is generally composed of four layers: the lens capsule 14, the subcapsular epithelium 16, the cortex 18, and the nucleus 20. The lens capsule 14, which includes an anterior capsule portion 22 and a posterior capsule portion 24, is a clear, membrane-like structure that encases the dense cellular elements of the lens 12. The lens capsule 14 is elastic, permitting the lens 12 to accommodate or change shape upon relaxing and contracting the zonular ligaments 26 attached to opposed ends of the lens capsule 14. The subcapsular epithelium 16 is a monolayer of epithelial cells beneath the anterior capsule portion 22 that produce lens fibers making up the lens cortex 18 and nucleus 20.

As described in more detail below, in some ophthalmologic procedures it is desirable to deliver various agents to the crystalline lens 12. This permits agents to be delivered directly to a site requiring evaluation and/or therapy. Use of a conduit to deliver agents to the lens allows the agents to be selectively located within the lens, and also allows the agents to be confined or localized within the lens. Thus, agent delivery via a conduit provides a method to deliver localized therapy. As one example, an increased concentration of a therapeutic and/or diagnostic agent may be introduced to the lens. Because of its localization and confinement within the lens, there is less of a concern for distribution to other ocular structures or for systemic distribution with concomitant toxicity, side effects, drug-drug interaction, etc. As another example, a vector containing a therapeutic gene may be introduced to the lens. Because of its localization and confinement within the lens, there is less of a concern for release or dissemination of the gene, etc., beyond the site of delivery.

As illustrated in FIG. 1A, a fine conduit 28 is used to introduce agents, shown schematically at 30 in FIG. 1A, into the lens 12 with a minimized risk of inducing cataract formation. In some ophthalmologic applications, it may be necessary to precisely locate the agents 30 within one or more of the various layers of the lens 12, such as the lens capsule 14 and/or the subcapsular epithelium 16. Introduction of agents 30 includes, but is not limited to, a therapeutic gene or portion thereof, a regulatory gene or portion thereof, a gene inducer or promoter, a gene inhibitor, an oligonucleotide, gene vectors, drugs, etc., with configuration or formulation of any of the above using nanotechnology, as known to one skilled in the art.

As used herein, a conduit 28 generally refers to any structure that defines an enclosed passageway through the structure. As examples, the conduit 28 may be or may include a needle, a cannula, a tube (e.g. glass, plastic, metal) or any other structure known to one skilled in the art that includes such a passageway. In one embodiment, conduit 28 provides passage for the agents 30 into the lens 12 proper, or into a specific portion of the lens 12, via a self-sealing aperture 32 of the lens capsule 14 caused by a fine gauge portion or tip 34 of conduit 28. A fine gauge portion or tip 34 is generally of 30 gauge diameter or higher (smaller diameter), with the higher the gauge, the thinner the needle. Such a delivery system minimizes, reduces, or prevents cataract formation. While not bound to a particular theory or mechanism, the reduced size of entry minimizes trauma to the lens capsule 14.

One example of such a conduit 28 in accord with the invention is a fine gauge needle. While needle size refers to both the length and gauge (thickness) of the needle, it is the needle gauge that is sufficiently thin for use in the inventive method. In one embodiment, a needle having a 30 gauge diameter (0.31 mm outer diameter) or higher (smaller outer diameter) may be used. Needles having a high gauge diameter (e.g. a 42 gauge diameter, 0.14 mm outer diameter) are commercially available, for example, Hamilton Company (Reno Nev.); Popper and Sons, (New Hyde Park N.Y.); synergetics™ inc. (St. Louis Mo.); Alcon (Houston Tex.); Bausch & Lomb (Rochester N.Y.) and other medical supply companies. Non-limiting examples include a 39 gauge rigid microinjection cannula straight, a 42 gauge rigid microinjection cannula straight, a 39 gauge rigid microinjection cannula angled, a 42 gauge rigid microinjection cannula angled, a 39/21 gauge rigid microinjection cannula straight with a 21 gauge shaft, a 42/21 gauge rigid microinjection cannula straight with a 21 gauge shaft, a 39/21 gauge rigid microinjection cannula angled with a 21 gauge shaft, and a 42/21 gauge rigid microinjection cannula angled with a 21 gauge shaft.

Another example of such a conduit 28 is a microcapillary pipette available for intracellular/extracellular microinjection. The microcapillary pipette is generally formed from a capillary tube, which generally ranges in size between 1-2 mm outer diameter and may be made from borosilicate glass or other suitable materials such as aluminosilicate or quartz. Such capillary tubes may be obtained commercially, or may be self-manufactured, e.g., formed from glass capillary tubes. To form the microcapillary pipette, the capillary tube is held in a device (termed a pipette puller, e.g., Sutter Instrument, Novato Calif.; Tritech Research, Los Angeles Calif.) and a portion of the tube is heated to the softening point of the particular material. Once the softening point is reached, a pulling force is applied to each end of the capillary tube, thinning the tube along the softened portion so as to have a desired diameter. Depending on a number of variables, such as capillary material type, temperature, and pulling force among others, numerous types and diameter sizes may be formed. This technique may, for example, be used to produce microcapillary pipettes with tip diameters of 0.02 μm. Those of ordinary skill in the art will recognize that the microcapillary pipettes may also go through additional processing steps, such as a beveling process to the tip 34 to facilitate penetration and/or agent delivery into the lens 12.

A number of devices that contain or are capable or containing agent may be coupled to the conduit 28. In one embodiment, the conduit 28 is coupled to a standard syringe containing the agent 30. The syringe may be calibrated to indicate the volume of agent 30, facilitating accurate dosage of agent injected into the lens 12. For example, a fine gauge needle or a microcapillary pipette operatively coupled with a commercially available syringe (e.g., Hamilton Company, Reno Nev.) may be used to introduce an agent 30 into the lens 12.

In one embodiment, a patient is prepared for the procedure with a local anesthetic, which may be injected, applied topically, etc. Under an operating microscope, a physician penetrates the lens capsule 14 with the tip or end 34 of conduit 28, such as the tip of a needle or micropipette as previously described, at one or more selected lens locations to deliver the agent. The choice of site(s) depends upon a variety of factors, such as the particular ophthalmologic treatment, location of pathology, lens size (child vs. adult), physician preference, etc. For example, as shown in FIG. 1A, the tip 34 of conduit 28 may be positioned within the cortex 18. The invention, however, is not so limited, and the particular length of the conduit 28 may be selected to facilitate positioning within the nucleus 20, the subcapsular epithelium 16, or the cellular layers that make up the lens capsule 14 (shown in phantom in FIG.1A). Moreover, the invention is not limited to penetration of the lens capsule 14 via the anterior capsule portion 22. In one embodiment, the conduit 28 may also penetrate the lens capsule 14 via the posterior capsule portion 24. In one embodiment, more than one conduit 28 may be introduced in a single procedure. Ultrasound may be used to visualize placement of tip 34 or conduit 28 within the lens.

Agent 30 is introduced into the lens 12, either directly through the tip 34 of conduit 28 or through a syringe barrel or other device coupled to conduit 28 (e.g., by manually depressing a plunger on the syringe). A conduit 28 and syringe, as described above, may be used for ophthalmologic therapies where precise positioning of the tip 34 of conduit 28 within lens 12 is not required. Alternatively, the position of the tip 34 of conduit 28 within the lens 12 may be secured by, for example, a biologically compatible sealant, adhesive, etc.

In another embodiment, a microinjection system may be used to introduce agent 30 into the lens 12 in a controlled and precise manner. Such a microinjection system generally includes a microinjector coupled to conduit 28 to finely control the amount or volume of agent 30 being dispensed through the conduit 28, and a micromanipulator to finely control the position of the tip 34. As is known in the art, different types of microinjectors are commercially available (e.g., Eppendorf AG, Hamburg, Germany; Tritech Research, Los Angeles Calif.). Microinjectors include syringe-based microinjectors, pneumatic or oil based microinjectors, and motor-driven microinjectors. The microinjectors are configured to control the amount or volume of agent 30 through the conduit 28. For example, a syringe-based microinjector generally includes a support or base, and a connecting member adapted to receive the barrel of a syringe. The end of the syringe is coupled to the conduit 28 through which the agent 30 will be dispensed. In one embodiment, a needle is coupled to the end of the syringe. In another embodiment, a micropipette is directly or indirectly coupled to the end of the syringe. The microinjector further includes an actuator that cooperates with the syringe or the plunger of the syringe such that displacement of the actuator causes the agent 30 to be dispensed through the conduit 28.

In one embodiment, the actuator is manually manipulated. To this end, the microinjector typically includes a screw with a handle or knob on one end. Movement of the knob in one direction, such as the clockwise direction, will advance the actuator toward the syringe and thereby dispense the agent 30 in the syringe through conduit 28. In a similar manner, movement of the knob in an opposite direction, such as the counterclockwise direction, will retract the actuator away from the syringe. Thus by manipulating the knob, the amount or volume of agent 30 dispensed through conduit 28 may be controlled. As those of ordinary skill in the art will recognize, the movement of the actuator may be controlled in other ways. Furthermore, those of ordinary skill in the art will recognize that pneumatic or motor driven microinjectors are available that also allow precise amounts or volumes of agent 30 to be dispensed.

The microinjection system may include a micromanipulator. The micromanipulator includes a connecting member adapted to couple to the conduit 28. The micromanipulator is configured such that the connecting member, and thus the conduit 28, may be moved in one or more directions. In one embodiment, a three-axis micromanipulator is used to control the position of the connecting member in three-dimensional space. In one aspect, the micromanipulator may be manually operated. To this end, the micromanipulator may include three handles that control movement of the connecting member along the three mutually exclusive axes. Thus, by manipulating the three handles, the position of conduit 28 may be controlled so as to accurately dispense agent 30 at a particular depth and into a particular site or location within lens 12. As those of ordinary skill in the art will recognize, the movement of the connecting member may be controlled in other ways. For example, instead of manual handles, the micromanipulator may include stepper motors that are coupled to a controller. The controller then actuates the stepper motors to move the connecting member in three-dimensional space to a desired location. The controller may include a joy stick such that movement of the joy stick in a certain direction causes movement of the connecting member in a corresponding direction. Micromanipulators of the type described above are commercially available (e.g., Eppendorf AG, Hamburg Germany; Tritech Research, Los Angeles Calif.).

In any of the above-described embodiments, the placement of tip 34 and/or delivery agent within the lens may be verified by ultrasound visualization.

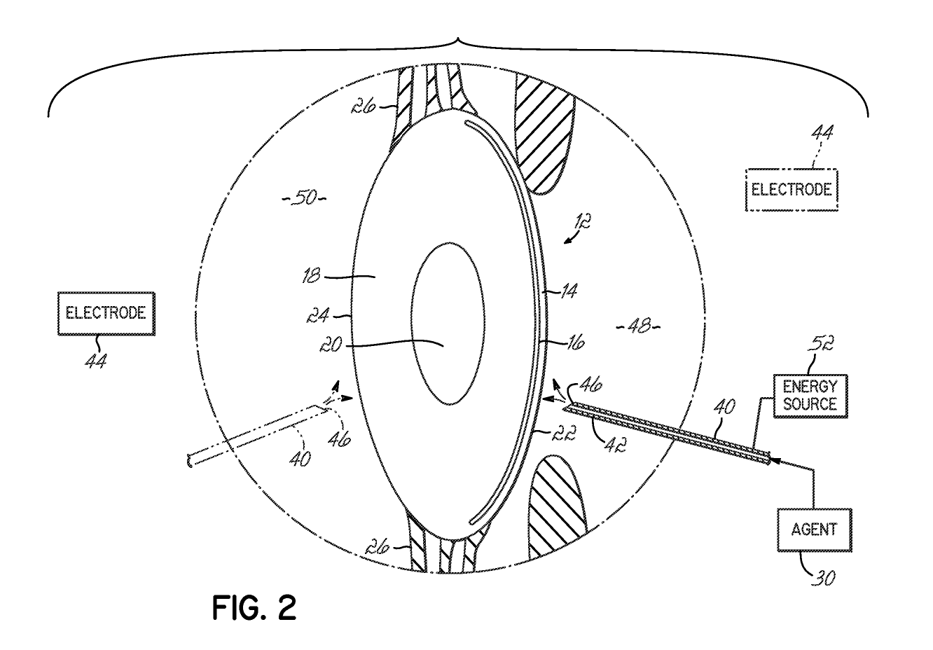

In another embodiment, as shown in FIG. 2 in which like reference numerals refer to like features in FIGS. 1 and 1A, a conduit 40 may be used to introduce agents into the lens 12 without penetration of the lens capsule 14. In particular, agents 30 may be delivered to the lens 12 using electromotive drug administration, also referred to as iontophoresis, by passing a small electrical current through the eye 10. In this embodiment, in addition to providing a passageway through which agents 30 may be introduced into the eye 10, the conduit 40 may further operate as an electrode 42, i.e., an anode and/or cathode depending upon the charge state of the agents 30. An electrode 44 of opposite polarity (cathode and/or anode) may be positioned at a site opposite conduit 40 so that lens 12 is disposed between the two electrodes 42, 44 and along an electrically conductive path.

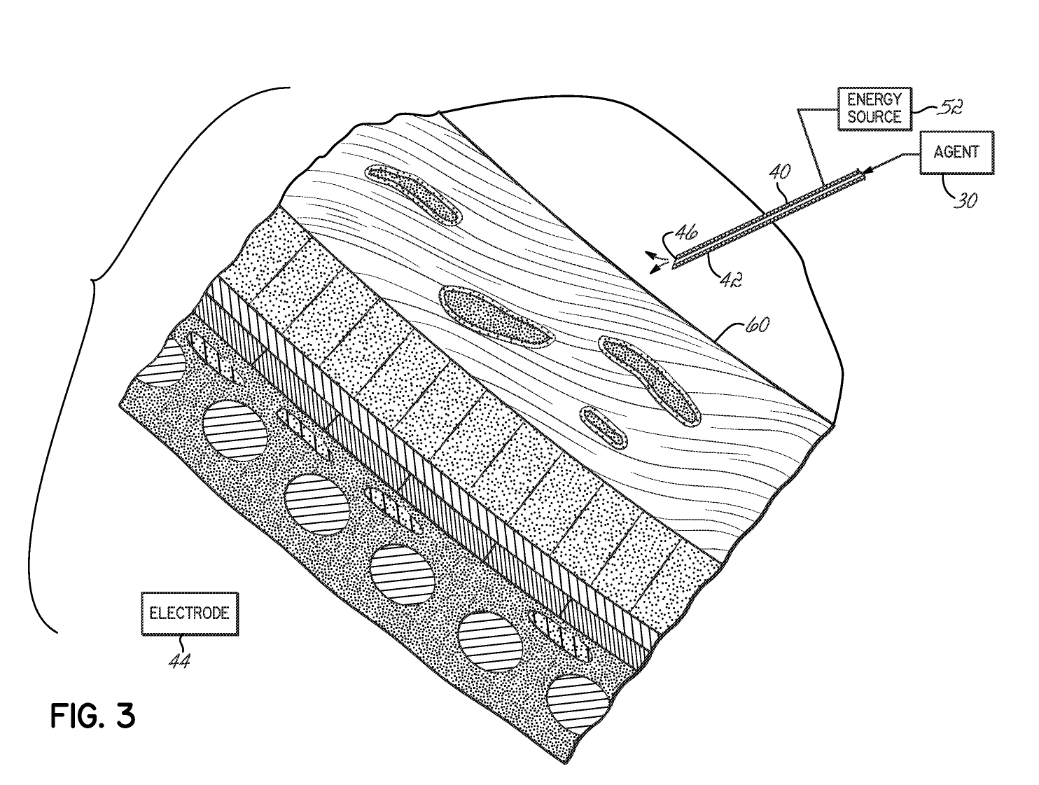

The electrode 44 positioned opposite the conduit 40 may be positioned within the body, such as behind the eye 10. Alternately, electrode 44 may be positioned outside the body of the patient. In one embodiment, electrode 44 may be positioned behind the patient's head. For example, such positioning of the electrode 44 may be used when the conduit 40 is positioned in the eye 10 and adjacent the anterior capsule portion 22, such as via the anterior chamber 48. When the conduit 40 is positioned in the eye 10 but adjacent the posterior capsule portion 24, such as via the vitreous cavity 50, electrode 44 may be positioned on or near the forehead of the patient (illustrated in phantom in FIG. 3). Those of ordinary skill in the art will recognize the appropriate location of electrode 44, depending on the position of the conduit 40, so as to ensure delivery of agents 30 to the lens 12 using iontophoresis.

The flow of current may be regulated externally to the eye 10 by a suitable energy source 52. When current is applied, an electrical potential difference is generated between the two electrodes 42, 44, facilitating movement of agents 30 away from conduit 40, toward electrode 44, and into the lens 12. Such administration may permit a relatively high concentration of agents to be delivered to the lens. The dose of agents 30 delivered to the lens 12 depends upon the current and duration selected. In one embodiment, a current between about 0.5 mA and about 4 mA is applied for between a few seconds to about 20 min. Those of ordinary skill in the art will recognize that the current and/or time duration may be manipulated so as to deliver the agents 30 into selected portions of the lens 12. For example, the longer the time duration, the deeper within the lens 12 agents 30 are capable of penetrating. Thus, agents 30 may be delivered to selective portions of the lens 12, such as the lens capsule 14, subcapsular epithelium 16, cortex 18, and/or nucleus 20 through these controllable parameters. Iontophoresis itself has no side effects and there is no pain associated with drug administration using this methodology.

In one embodiment, a patient is prepared for the procedure with a local anesthetic. Under an operating microscope, a physician penetrates the eye 10 at one or more locations so as to position the tip 46 of conduit(s) 40 adjacent the lens 12. The conduit tip 46, however, does not penetrate the lens capsule 14. As before, the choice of site(s) depend upon a variety of factors, such as the particular ophthalmologic treatment, location of pathology, lens size, physician preference, etc. As shown in FIG. 2, the conduit tip 46 may be positioned in the anterior chamber 48 and adjacent the lens 12. Other positions, however, such as approaching the lens 12 from the vitreous cavity 50, are also possible. Ultrasound may be used to visualize placement of conduit tip 46 adjacent lens 12.

Drugs are then introduced into the eye 10 immediately adjacent the lens 12, either directly through the tip of conduit 40 or through a syringe barrel or other device coupled to conduit 40. As in the previous embodiment, a microinjection system may be used to introduce the agents 30 adjacent the lens 12 in a controlled and precise manner. Prior to, subsequent to or essentially simultaneous with the delivery of the agents 30 via conduit 40, the electrodes 42, 44 may be energized so as to direct the agents 30 into the lens 12.

In one embodiment, one or more agent(s) 30 that influences the shape of the lens 12 may be administered into or adjacent lens 12 depending on whether a penetrating or non-penetrating methodology is utilized. Such an agent, or combination of agents 30, may be introduced at the anterior capsule portion 22 and/or the posterior capsule portion 24 to decrease or increase lens convexity. These agents 30 include synthetic and/or organic materials including collagens, mucopolysaccharides, glycosaminoglycans, liquid silicon, etc.

In one embodiment, an agent 30 that minimizes or prevents lens hardening, enhances or increase lens softening, and/or returns or enhances lens plasticity and/or elasticity may be administered into or adjacent lens 12 depending on whether a penetrating or non-penetrating methodology is utilized. As one example, an agent 30 that damages the cell membrane to enhance dissolution of lens fibers within the lens cortex 18 or the lens nucleus 20 may be administered. As another example, an agent 30 that renders the lens 12 more elastic is administered to an individual with presbiopia. As another example, an agent that reduces or prevents senescence may be administered to immortalize cells or cell types within the lens.

In one embodiment, an agent 30 that inhibits cell proliferation of the lens epithelium 16 may be administered into or adjacent lens 12 depending on whether a penetrating or non-penetrating methodology is utilized. Such an agent 30 may reduce, delay, or prevent opacification of the anterior and/or posterior capsule portion 22, 24, respectively, after cataract surgery. As one example, a pharmacological compound and/or a vector carrying a gene modifying the survival of the lens epithelium 16, or causing it to produce compounds which enhance survival of the lens epithelium 16 or other lens fibers, may be administered. Such agents 30 may be administered alone, or in combination with antiproliferative agents to reduce capsular opacification and cell proliferation. These antiproliferative agents are known to one skilled in the art and include, but are not limited to, methotrexate, cyclophosphamide, ifosphamide, 5-fluorouracil, 5-fluorouridine, cytarabine, bleomycin, mitomycin-c, etc.

In one embodiment, one or more antioxidants are provided to the lens using the inventive methods. It is known that glucose metabolism and its associated effect on redox potential have a role in crystalline lens alteration; this may induce oxidative damage. Thus, antioxidant agents (e.g., drugs with radical scavenging properties, vitamin E, vitamin C, carotenes, lutein, zeaxanthin, molybdenum, retinol, etc.) may be provided into or adjacent the lens.

Any of the above agents may be formulated. Genes may be provided in vectors (e.g., entrained, targeted, encapsulated, etc.). Drugs may be formulated as nanoparticles or nanocrystals of pharmaceutically active compounds, and/or nanoscale dispersions, encapsulations, and emulsions (e.g., to limit or prevent aggregation or reaggregation of crystals, to incorporate a stabilizer, etc). The drugs may be combined with albumin or another non-toxic solvent to form nanoparticles in a solvent-free formulation of a toxic drug. The drugs may be formulated as sugar-derived nano compounds that may shield proteins and small molecules from rapid breakdown. The drugs may be rendered more soluble in a nanocrystal formulation by decreasing drug particle size and hence increasing the surface area thereby leading to increased dissolution. These techniques are known to one skilled in the art as disclosed in, for example, U.S. Pat. Nos. 6,822,086; 6,753,006; 6,749,868; 6,592,903; 6,537,579; 6,528,067; 6,506,405; 6,375,986; 6,096,331; 5,916,596; 5,863,990; 5,811,510; 5,665,382; 5,560,933; 5,498,421; 5,439,686; and 5,362,478; and U.S. Patent Application Ser. Nos. 10/106,117; 60/147,919; and 08/421,766, each of which is expressly incorporated by reference herein in its entirety.

The invention will be further appreciated with respect to the following non-limiting example.

### EXAMPLE

A 33 gauge needle penetrated the lens capsule and epithelium of a New Zealand white rabbit. The tip of the needle was positioned within the cortex. Fluorescein dye, contained within a barrel of the syringe coupled to the needle, was injected into the lens (0.1 μl-3 μl) by manual depression of the syringe plunger.

At eight weeks post-injection, the fluorescein solution had uniformly stained the entire lens, detectable upon visual observation. This indicated that the solution had disseminated from the single site of injection throughout the entire compact dense cellular material of the lens. Moreover, at eight weeks post-injection, there was no cataract formation observed upon ophthalmologic evaluation. There was minimal scarring and no evidence of permanent egress or ingress of fluid from inside or outside the eye at the injection site.

These data supported the likelihood that an agent, such as a vector containing a therapeutic gene, when injected into the lens, will be confined to the lens. Thus, any post-injection cell proliferation that may lead to cataract formation will be minimized or reduced.

The inventive methods are not limited to treatment of the crystalline lens but may be used to treat other ocular structures. Thus, other pathologies of the eye may benefit from the drug delivery methodologies of the invention. By way of example, the inventive method utilizing iontophoresis may be used to treat various pathologies of the retina 60 or other ocular structures (FIG. 1).

Diabetic retinopathy is a leading cause of blindness. Patients with diabetes mellitus have an absolute or relative lack of circulating insulin and, through a variety of factors, frequently present with vascular changes in the retina. These changes manifest in retinal microaneurysms, small hemorrhages, and exudates, and lead to the formation of scar tissue. New blood vessels may form around the optic disk (proliferative retinopathy). Over time, the cumulative results of such vascular effects lead to ocular pathologies which, ultimately, decrease vision in the diabetic patient. Thus, compositions and methods which reduce these vascular changes, or reduce their effects, improve the chances of a diabetic patient either maintaining vision, or at least slowing loss of vision.

Macular degeneration, also called age-related macular degeneration, is a pathological condition that results in proliferation of new blood vessels in the subretinal area. While the presence of the new vessels themselves is not problematic, the new vessels leak blood and other serous fluid that accumulate in surrounding spaces. It is this fluid accumulation that leads to visual impairment. For example, in the retina, both the large vessels and the capillaries normally have intact vessel walls. In the choroid, the large vessels normally have intact vessel walls, but the capillary walls or membranes contain fenestrations or openings. Any endogenous or exogenous fluid present in these capillaries, for example, blood, serous fluid, solubilized drug, etc. will leak outside the vessels and into the surrounding area. The accumulation of fluid can result in serous and hemorrhagic detachment of the retinal pigment epithelium and neurosensory retina, and can lead to loss of vision due to fibrous deform scarring. Patients with an early stage of age-related macular degeneration can be diagnosed by the presence in the eye of abnormal clumps of pigments, termed drusen, which are dead outer segments of photoreceptor cells under the retinal pigment epithelium. The presence of large, soft drusen in the eye indicates a pre-stage of exudative age-related macular degeneration, and places these patients at higher-than-average risk for developing neovascularizations, especially if one eye is already affected.

Retinitis pigmentosa is a general term that encompasses a disparate group of disorders of rods and cones, which are the sensory structures in the retina. While retinitis pigmentosa is a genetic disorder, and is not an inflammatory process, one manifestation of the disease is the presence of irregular black deposits of clumped pigment in the peripheral retina. Thus, there is likely at least some immune component to retinitis pigmentosa.

In yet another embodiment, as shown in FIG. 3 in which like reference numerals refer to like features in FIG. 2, the conduit 40 may also be used to introduce agents into the retina 60 without penetration of the retina 60. The conduit 40 as well as the electrodes 42, 44 operate in a substantially similar manner as that described above to deliver agents to the retina 60. In particular, in operation, a patient is prepared for the procedure with a local anesthetic. Under an operating microscope, a physician penetrates the eye 10 at one or more locations so as to position the tip 46 of conduit(s) 40 adjacent the retina 60. The conduit tip 46, however, does not penetrate the retina 60. As shown in FIG. 3, the conduit tip 46 may be positioned in the vitreous cavity 50 and adjacent the retina 60. Ultrasound may be used to visualize placement of conduit tip 46 adjacent retina 60.

Drugs are then introduced into the eye 10 immediately adjacent the retina 60, either directly through the tip of conduit 40 or through a syringe barrel or other device coupled to conduit 40. A microinjection system may also be used to introduce the agents 30 adjacent the retina 60 in a controlled and precise manner. Prior to, subsequent to or essentially simultaneous with the delivery of the agents 30 via conduit 40, the electrodes 42, 44 may be energized so as to direct the agents 30 into the retina 60. As before, the current and/or time duration may be manipulated so as to deliver the agents 30 into selected portions of the retina 60. For instance, the longer the time duration, the deeper within the retina 60 agents 30 are capable of penetrating.

Other variations or embodiments of the invention will also be apparent to one of ordinary skill in the art from the above figures, description, and example. Thus, the forgoing embodiments are not to be construed as limiting the scope of this invention.

What is claimed is:

1. An ocular drug delivery method comprising:
penetrating an eye with a conduit so as to position an opening of the conduit adjacent an ocular structure,
introducing a drug in proximity to the ocular structure without penetrating the ocular structure through the opening of the conduit, and
directing the drug into the ocular structure using iontophoresis.

2. The method of claim 1 wherein the ocular structure is selected from the group consisting of a lens, a retina, and combinations thereof.

3. The method of claim 1 wherein directing the drug into the ocular structure using iontophoresis further comprises configuring the conduit to operate as a first electrode, positioning a second electrode in relation to the first electrode to facilitate movement of the drug into the lens, and energizing the electrodes to cause a current to flow between electrodes.

4. The method of claim 3 wherein the second electrode is positioned outside the body.

5. The method of claim 3 wherein the second electrode is positioned behind the head or on the forehead.

6. The method of claim 3 further comprising varying at least one of current magnitude or current duration to control drug delivery to the ocular structure.

7. The method of claim 1 further comprising performing ultrasound to visualize placement of the conduit in the eye.

8. The method of claim 1 wherein the drug is in a nanotechnology formulation.

9. The method of claim 1 wherein the drug is an oligonucleotide.

10. The method of claim 1 wherein the drug is at least a portion of a therapeutic gene in a vector.

11. The method of claim 10 wherein the vector is a virus or a plasmid.

12. The method of claim 1 wherein the drug regulates at least one of cell proliferation, cell mortality, or cell integrity.

13. The method of claim 12 wherein regulation is inhibitory or stimulatory.

14. The method of claim 1 wherein the ocular structure is a crystalline lens and the conduit is positioned adjacent an anterior lens capsule or a posterior lens capsule.

15. An apparatus for delivering an agent to the eye, the apparatus comprising
a conduit capable of penetrating the eye and having a passageway for delivery of the agent to the eye; and
a power source adapted to be coupled to the conduit,
wherein the conduit is configured to operate as a first electrode for iontophoresis delivery of the agent when coupled to the power source.

16. The apparatus of claim 15 further comprising a second electrode.
